(19) United States
(12) Patent Application Publication
(10) Pub. No.: US 2022/0062097 A1
(43) Pub. Date: Mar. 3, 2022

(54) PENILE HOLDER

(71) Applicant: Metro Men's Health, Salt Lake City, UT (US)

(72) Inventors: Warren Fairbanks Kirk, Millcreek, UT (US); Troy Armstrong Lunt, Millcreek, UT (US)

(21) Appl. No.: 17/009,716

(22) Filed: Sep. 1, 2020

(51) Int. Cl. A61H 23/00; A61F 5/41

(52) U.S. Cl. CPC A61H 23/008; A61F 5/41; A61H 2201/0184; A61F 2005/417; A61H 2201/165; A61H 2205/087

(57) ABSTRACT

A penile holder device includes a base, a riser attached to the base at a central portion of the base, the riser including a holder support and an open channel through an upper portion of the riser and a holder having an attachment portion complementary to the holder support in the riser. A holder portion extends horizontally from a top edge of the attachment portion. The attachment portion includes a slot and an attachment portion front edge is in a same plane as holder portion front edge. A locking mechanism can lock the holder in a chosen position relative to the riser. The holder can also tilt and the holder device can include a scale (configured, for example, in the riser) used to trigger a notification when a threshold amount of pressure is placed on the holder portion.

### TECHNICAL FIELD

This disclosure introduces a penile holder having improvements in the structure over prior holders to enable a doctor or technician to perform a wave therapy treatment for erectile dysfunction.

### BACKGROUND

FIGS. 1A-1C illustrates a prior art penile holder 100 that has been in the marketplace for use in wave therapy for erectile dysfunction (ED). The patient typically will sit on the edge of a table and the penile holder is positioned to enable the patient's penis to rest in a holder component 106 for the treatment. Sonic wave or shock wave therapy is a safe, comfortable and proven procedure for men to optimize sexual performance and to reverse the effects of ED due to poor blood flow. Wave therapy utilizes high frequency, low intensity sound waves to improve blood flow to the penis.

As shown in FIGS. 1A-1C, a base 102 of the prior holder is 13.375 inches wide, 3.5 inches deep and 0.5 inches in height. The height of a first support component 104 is 4.125 inches. A knob 106 is used with a screw to adjust the height of a penile holder component 108. The penile holder component 108 is 5.788 inches long. A second support component 110 is attached to the penile holder component and is used to adjust the height of the penile holder component 108.

There are several weaknesses to this prior art penile holder. First, given the human anatomy and often the structure of the patient table that the patient sits on, the size and adjustment capabilities of the penile holder 100 are not sufficient to position the holder component 108 in a comfortable and proper position. The penile holder 100 is also not strong enough to be used over time. Next, feature 112 shows an end portion of the holder portion 108 that hangs over the support 110 which is configured to slide along support 104. The overlap 112 can cause instability in the system. The wider portion of the support 110 is orthogonal to the direction of the holder portion 108, which can cause instability and limits its use. There is also only one degree of freedom in the adjustment capabilities of the penile holder 100. These and other deficiencies in the current penile holder design are addressed in the improvements described herein.

### BRIEF DESCRIPTION OF THE DRAWINGS

In order to describe the manner in which the various advantages and features of the disclosure can be obtained, a more particular description of the principles described above will be rendered by reference to specific embodiments thereof, which are illustrated in the appended drawings. Understanding that these drawings depict only example embodiments of the disclosure and are not to be considered to limit its scope, the principles herein are described and explained with additional specificity and detail through the use of the drawings in which:

FIGS. 1A-1C illustrate a prior art penile holder;

FIGS. 2A-2E illustrate an improved penile holder;

FIGS. 3A-3F illustrate components of the improved penile holder;

FIGS. 4A-4B illustrate a quick release mechanisms for a penile holder;

FIG. 5 illustrates a scale built into a penile holder for triggering a notification upon a threshold pressure being placed on the holder component;

FIGS. 6A-6C illustrate the holder portion;

FIG. 7 illustrates an example computing device architecture, in accordance with some examples of the present disclosure; and

FIG. 8 illustrates an example method.

### DETAILED DESCRIPTION

Certain aspects and embodiments of this disclosure are provided below. Some of these aspects and embodiments may be applied independently and some of them may be applied in combination as would be apparent to those of skill in the art. In the following description, for the purposes of explanation, specific details are set forth in order to provide a thorough understanding of embodiments of the application. However, it will be apparent that various embodiments may be practiced without these specific details. The figures and description are not intended to be restrictive.

The ensuing description provides example embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the application as set forth in the appended claims.

### Overview

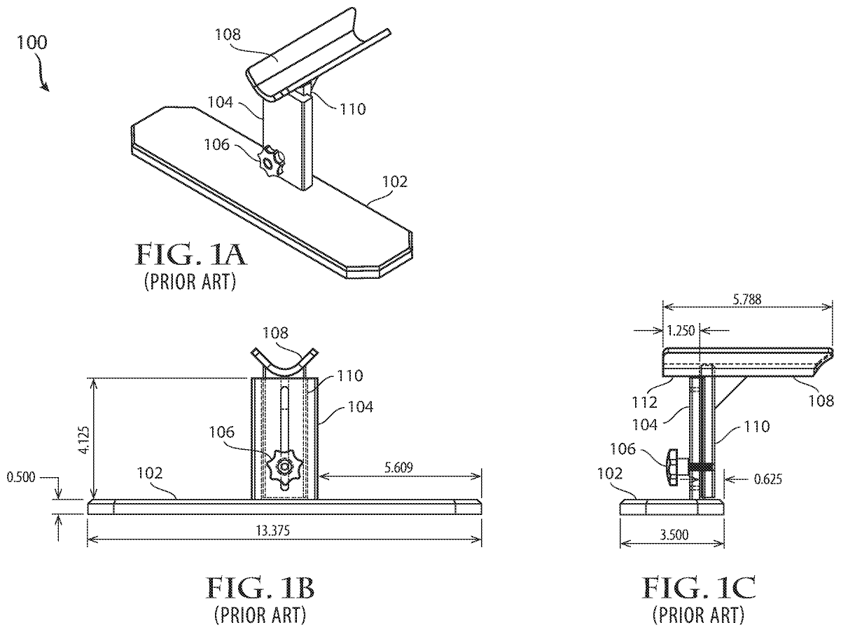

The present disclosure introduces a new penile holder that addresses one or more of the deficiencies in the prior art holder shown in FIGS. 1A-1C. The improved design provides more comfort and flexibility when using the penile holder for patients receiving ED wave treatment. Additional features are disclosed such as the ability to tilt the holder portion and the inclusion of a scale to gauge the pressure being placed on the holder portion during treatment. A notification component can instruct the medical personnel that a threshold pressure has been reached.

An improved penile holder device includes one or more of a base, a riser attached to the base at a central portion of the base, the riser including a holder support and an open channel through an upper portion of the riser and a holder having an attachment portion complementary to the holder support in the riser. The attachment portion can have a wider surface in a same plane as the direction along the length of a holder portion. The configuration of the attachment portion and the riser (which can be cylindrical in shape with a slot configured for the attachment portion to be inserted) can provide new capabilities for the penile holder. These features enable the holder portion to tilt, which capability was not possible in the prior art penile holder. The holder portion can extend horizontally from a top edge of the attachment portion. The attachment portion can include a slot and an attachment portion front edge is in a same plane as holder portion front edge. A locking mechanism can lock the holder in a chosen position relative to the riser. The holder can also tilt and the holder device can include a scale (configured, for example, in the riser) used to trigger a notification when a threshold amount of pressure is placed on the holder portion. The pressure can relate to how much pressure a doctor might use while providing treatment with a wave-producing medical tool.

Improvements also include new dimensions for the penile holder that increase the stability and flexibility of the system. The base can be approximately 15 inches wide and approximately 4 inches deep. In this case, “approximately” means the given length plus or minus 8% of that distance. The base can include a hole that is used to attach the riser to the base via a dowel. The riser can be configured to be approximately 8 inches high. A scale can be configured in some location in the device to trigger a notification upon a threshold pressure being pressed down on the holder. In one example, the scale is configured in the riser. The scale can be configured in the riser, for example, below the holder support.

The first member and the second member can include a quick release mechanism or a screw and a knob. Other mechanisms can be used to adjust at least one of a height and a tilt of the holder.

The holder attachment of the holder can be approximately 5 inches long. The holder portion can be approximately 7 inches long. The holder portion can be configured to be approximately 2.2 inches wide and approximately 1 inch tall.

The holder portion can have an interior surface having a first surface at an approximately 90 degree angle to a second surface. The holder portion can be attached to the attachment portion at a first end and the holder portion can include a second end having a convex surface having a depth of approximately 0.6 inches.

The above introduction and other improvements in the dimensions and configuration of the penile holder address the weaknesses in the prior art design. The new design provides greater stability, more comfort and flexibility, and can include a scale for identifying a threshold pressure during treatment. The new penile holder provides an overall improvement in the treatment experience for patients with ED.

### DETAILED DESCRIPTION

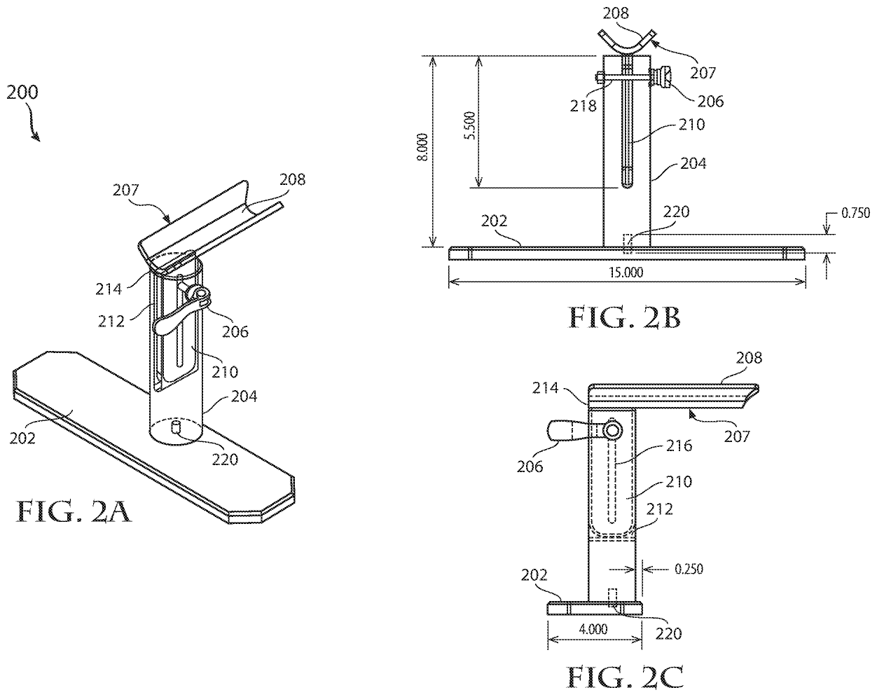

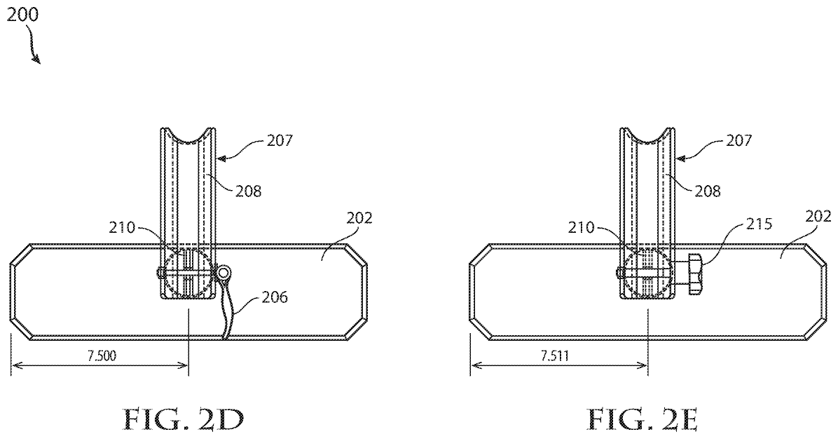

This disclosure now provides more detail regarding new structures as introduced above. FIGS. 2A-2E illustrate one example of the new penile holder design 200. An improved penile holder device 200 includes one or more of a base 202, a riser 204 attached to the base 202 at a central portion of the base 202, the riser 204 including a holder support 210 and an open channel 218 through an upper portion of the riser 204 and a holder 207 having a holder portion 208 and an attachment portion 210 complementary to the holder support 212 in the riser 204. The attachment portion 210 can have a wider surface in a same plane as the direction along the length of a holder portion 208. Configuring the wider surface of the attachment portion 210 to be in the same plane as the direction along the length of the holder portion 208 can provide greater stability plus a greater ability to cause the holder 207 to be raised up and tilted relative to the riser 204. Introducing the ability to tilt the holder portion 208 can improve the comfort of the penile holder for the patient during treatment because the holder portion 208 is not limited to a horizontal configuration. FIGS. 2A and 2D illustrate the structures of the attachment portion 210 and the holder portion 208.

In one example, the riser 204 is cylindrical in shape but could also have a rectangular or square cross-sectional shape. Other shapes are contemplated as well. The holder portion 208 can extend horizontally from a top edge of the attachment portion 210. The attachment portion 210 can include a slot 216 and an attachment portion front edge 214 can be in a same plane as holder portion front edge 214. This feature is illustrated in FIGS. 2A and 2D.

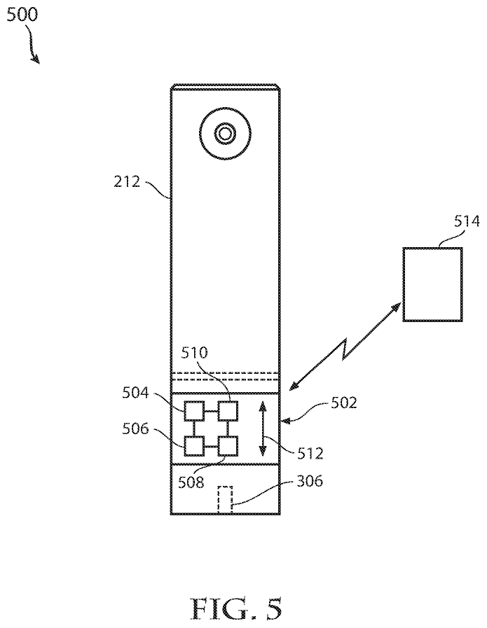

A locking mechanism 206 can lock the holder 207 in a chosen position relative to the riser 204. In one aspect, the device 200 can enable the holder portion 208 to be raised to height of approximately 12 or 12.5 inches. The holder 207 can also tilt and the holder device 200 can include a scale (configured, for example, in the riser or the base) used to sense the pressure on the holder portion 208 (due to treatment with the medical device) and trigger a notification when a threshold amount of pressure is placed on the holder portion 208. The scale will be introduced in more detail in connection with the discussion of FIG. 5.

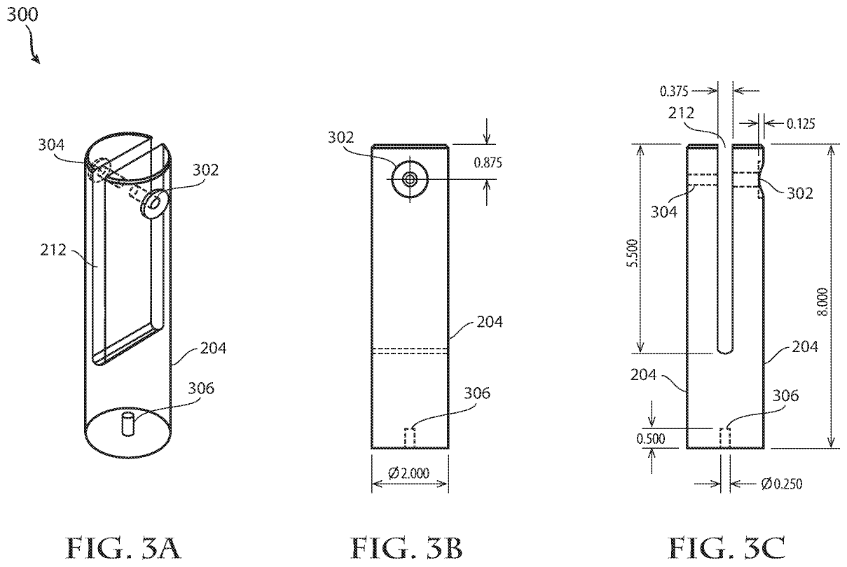

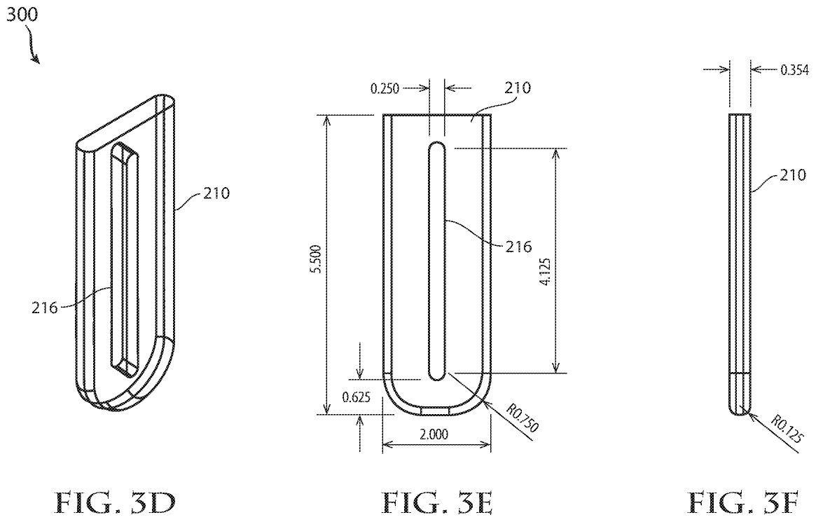

FIGS. 3A-3F illustrate additional features of the holder device 200 and focus on the structure of the riser 204 and the attachment portion 210. FIG. 3A illustrates the riser 202, a cavity 306 which can receive a dowel or other member for securing the riser 204 to the base 202. The holder support 212 is shown as a slot in the riser 204 that is approximately 5.5 inches in length. A tapped opening or hole 304, 302 is configured in an upper portion of the riser 204. FIG. 3D illustrates the attachment portion 210 which is configured to be attached to the holder portion 208. The attachment portion 210 includes a slot 216. A screw can be configured to be inserted through the hole 302, through the slot 216, and through the hole 304 and which can be used to adjustably secure the position of the holder portion 208 in a desired position including at a tilt. FIG. 3F illustrates a side view of the attachment portion 210 with the example dimensions of a width of approximately 0.35 inches and radius of curvature of approximately 0.125. The attachment portion 210 in FIG. 3E is shown as having a width of approximately 2 inches, a height of approximately 5.5 inches, a slot width of approximately 0.25 inches, a slot length of approximately 4.125 inches, and a distance between a lower portion of the slot 216 and a lower portion of the attachment portion 210 of a possibly 0.625 inches. A radius of curvature at the lower portion of the attachment portion 210 is shown to be at approximately 0.75 inches. All of these dimensions are shown by way of example and variations on each respective dimension can be included as within the scope of this disclosure. For example, the term “approximately” should be construed to mean the stated length plus or minus 8%. However, in other aspects, and a respective length can vary from 8% to 25% or even more.

Improvements also include the new dimensions that increase the stability of the system. The base 202 can be approximately 15 inches wide and approximately 4 inches deep. These dimensions are illustrated in FIG. 2B. The base 202 can include a hole or cavity that is used to attach the riser 204 to the base via a dowel 218 or other attachment mechanism. The riser 204 can be configured to be approximately 8 inches high. These changed dimensions from the prior art combine to improve the strength and stability of the medical device.

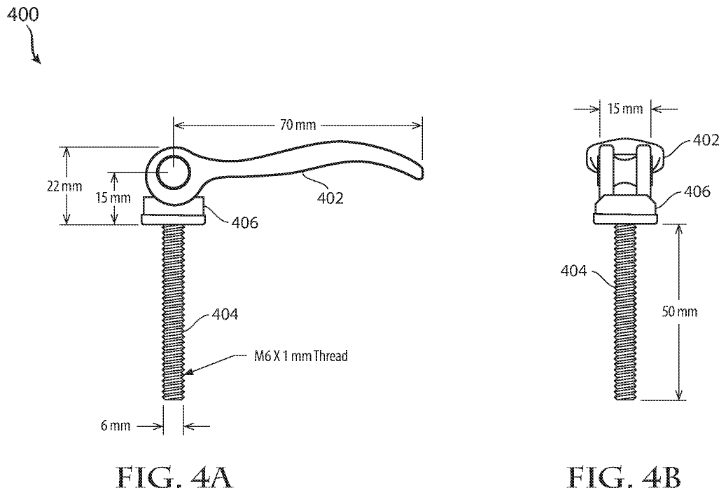

FIGS. 4A and 4B illustrate a quick release of a locking mechanism which can be used in connection with the openings 302, 304 in the upper portion of the riser 204 and the slot 216 of the attachment portion 210. As shown in FIG. 3C, the openings 302, 304 are configured near a top edge or a top portion of the riser 204. With respect to adjustably securing the holder portion 208 in a desired position relative to the riser 204, a first member 404 and the second member 402 can include a quick release mechanism 400 or a screw and a knob 214 as shown in FIG. 2F. Other mechanisms can be used to adjust at least one of a height and a tilt of the holder portion 208.

FIG. 5 illustrates a riser 500 that can include a scale 502 that can be configured to trigger a notification upon a threshold pressure being pressed down on the holder portion 208. For example, as a doctor or technician uses a wave-producing medical tool to apply the treatment to the patient, it can be important to know what pressure level is being applied to the patient. A scale can be incorporated into the penile holder 200 to sense or detect the pressure level and provide feedback to the doctor when a threshold pressure value is achieved.

In one example, the scale 502 is configured in the riser 500, for example, below the holder support 212. The scale can also be configured in the base 202, the holder 207, or any other location within the penile holder 200. As shown in FIG. 5, the scale 502 can include a number of electrical and mechanical components. For example, a controller 504 can be attached to a battery 506 that can include a sensor 508 that can receive signals from in mechanical or electrical scale component 512. Any type of mechanical or electrical scale can be used in connection with the device 200 for the following purpose. When treating a patient using the wave technology disclosed herein, a doctor or technician will apply sound waves to the penis positioned within the holder portion 208. A certain amount or range of pressure may be desirable from a medical standpoint to ensure that the proper amount of energy is applied by a wave-producing medical device (not shown). A threshold amount or range can be programmed or input into the controller 504. A memory can be included as part of the component shown for the scale 504. The scale 502 can be incorporated into the holder device 200 for the purpose of gauging and providing the doctor with feedback regarding the proper amount of force exertion or pressure being used. Any type of scale, such as a spring scaled, a mechanical scale, a micro-balance or nano-balance scale which provide more precise measurements, can be used. Feature 510 shown in FIG. 5 represents an output device that can include, as instructed by a controller 504, a speaker, a display, a light, a haptic device, a multi-model device, a wireless communication module for transmitting a signal to a separate device, and so forth.

Generally speaking, the holder device 200 can include a scale 502, which can be operable and configured to determine whether a threshold amount of pressure or force has been provided via the use of the wave-producing medical device. When the threshold pressure is experienced by the scale, a sensor 508 can cause or trigger a notification to the doctor or technician which can be provided via the output component 510. Thus, as a doctor or technician is treating a patient, a light, or a sound, or a notification from a mobile device such as a smart phone or laptop or other medical device, can indicate that the proper amount of pressure has been applied using the wave-producing medical device. Such feedback can enable the treatment to be more consistent and effective. Wireless communication components to enable a wireless link or wireless communications between the scale 512 and a remote device 514 can be included in the scale 512 structure.

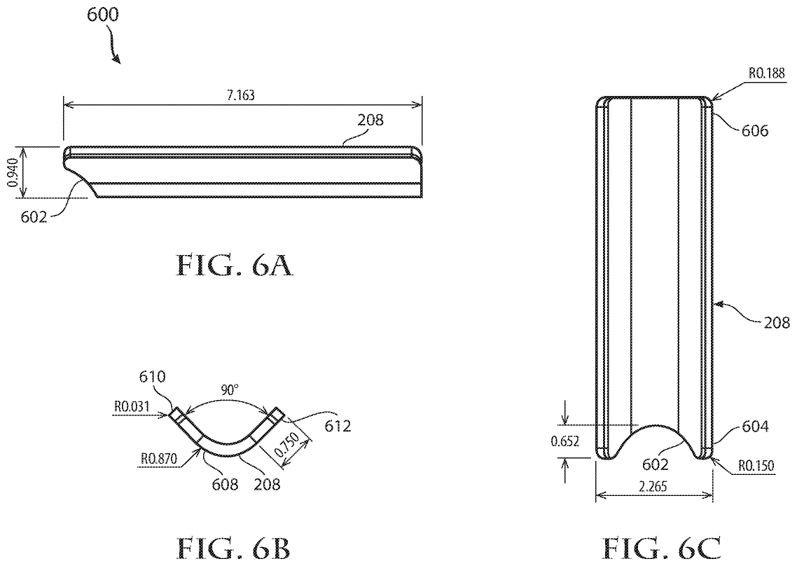

FIGS. 6A-6C illustrate additional details of the structure of the holder portion 208. The holder portion 208 of the device 200 can have a length of approximately 7.163 inches and a height of approximately 0.94 inches as shown in FIG. 6A. A convex surface 602 can be included which as is shown in FIGS. 6A and 6C illustrate an end of the holder portion 208 which is cut back or configured to provide a more comfortable use of the holder device 200 for the patient. FIG. 6C shows an example width of the holder portion 208 to be approximately 2.265 inches and the depth of the convex surface 602 at approximately 0.652 inches from the end of the holder portion 208. An interior surface can have a first surface at an approximately 90 degree angle to a second surface as shown in FIG. 6B. The holder component can be attached to the attachment portion 210 at a first end.

A radius of curvature associated with various corners and surfaces are also shown by way of example. For example, FIG. 6B illustrates an example of a radius of curvature for the bottom portion 608 of the holder portion 208 to be approximately 0.87 inches. A radius of curvature 610 for a top edge of the holder portion 610 is shown at 0.031 inches. A radius of curvature for an end 604 of the holder portion 208 can be approximately 0.150 inches and a radius of curvature of a second end 606 of the holder portion 208 can be approximately 0.188 inches. These are all given as example dimensions and variations are also contemplated.

The above introduction and other improvements in the dimensions and configuration of the penile holder 200 address the weaknesses in the prior art design. The new design provides greater stability, more comfort and overall an improvement in the treatment experience for patients with ED.

In another example, a device includes a base 202 and a riser 204 attached to the base 202 at a central portion of the base. The riser 204 can include a holder support 212 and an open channel 218 through an upper portion of the riser 204. A holder 207 can have an attachment portion 210 complementary to the holder support 212 in the riser 204 and a holder portion 208 that extends horizontally from a top edge of the attachment portion 210. An attachment component 206 can adjustably attaches the holder 207 to the riser 204. An optional scale 502 can trigger a notice upon a threshold pressure being pressed on the holder or holder portion.

The scale 502 further can include one or more of a battery 506, a controller 504, a sensor 508 and an electronic notification component or output component 510. A wireless communication component can be included as well. The notice can include one or more of an audio notice, a visual notice, a tactile notice, and a multi-modal notice. The scale 502 can further include a transmission component configured to communicate wirelessly with a remote device 514 such as a mobile phone or server system over a network. The communication can be achieved through any wireless protocol such as BlueTooth™, WiFi, cellular, 5G, or any protocol for communicating data. Thus, a doctor or technician could receive a notification (sound, text, tactile output, etc.) on a mobile phone or a device held by the doctor or technician. The wave-producing medical device can also be configured to receive information about the amount of pressure such that the notification that a threshold pressure has been met can be provided to the doctor or technician right on the wave-producing medical device. Thus, the scale 502 and its components can be configured to communicate with the other medical tool used to provide the treatment. In one aspect, the function of the wave-producing medical device could be coordinated with the scale 502 data. For example, the waves that are produced can be adjusted or cut off relative to the sensed pressure on the holder portion 208.

The scale 502 can further include a controller 504 that can be configured to adjust the threshold pressure on an individual basis. The adjustment can be made through the use of an app on a mobile device that inputs data about the individual patient. The medical wave-treatment tool can receive patient data, or the scale 502 can have a user interface that receives input about the patient. Then, the proper threshold can be identified and applied for the treatment for that patient.

The type of medical device used to treat ED is not relative to the present disclosure. For example, the tool may use sound waves or electromagnetic waves. Other medical treatments may be used as well in connection with the disclosed penile holder 200. Because the wave-producing tool might also be electromagnetic rather than sound-based, the threshold pressure levels might need to be adjusted based on the type of treatment. Thus, the threshold pressure level that triggers a notification can vary based on the type of medical device being used for the ED treatment.

Embodiments can be claimed from the standpoint of the holder device 200, the wave producing medical device, a combination of both, or a separate mobile device or server as described herein. For example, an application or an “app clip” as introduced by Apple Computers, or through a browser or other user interface, can be configured on a mobile device and steps are performed by that mobile device can be covered as it receives data, interacts with the holder device 200, receives notification or triggers, and so forth. All steps or operations that might be performed by the holder device 200 and particularly with respect to the built-in scale 502, are covered as within the scope of this disclosure. For example, receiving sensor readings, generating data, evaluating a weight experienced by the scale and determining whether a threshold pressure has been met, and taking other steps such as communicating or transmitting data from the holder device 200 to a remote device 514 are all covered as within the scope of this disclosure. These various steps can be claimed from the standpoint of the holder device 200, from the standpoint of a remote device 514 or from the standpoint of both devices operating together. The remote device 514 can represent a mobile phone, a remote device, a server over the Internet, a medical tool, and so forth.

In one aspect, the data can be gathered for each individual treatment through sensors that are part of the scale 502. For example, data such as how long the treatment lasts, the weight or pressure applied during the treatment, information about the patient, information about the signals used in the wave-procedure, and so forth can be gathered, aggregated and used for improving the treatment process. For example, in accordance with guidelines for managing patient information, such data can be aggregated, and otherwise, and used to provide guidance regarding additional treatments such that through one or more of the wave-treatment medical device, the scale 502 as part of the holder device 200, the doctor can be guided with respect to how to perform the treatment based on historical data which can be linked to success rates. Machine learning tools of any type can also be used to evaluate the data and to provide guidance or suggestions regarding treatment procedures.

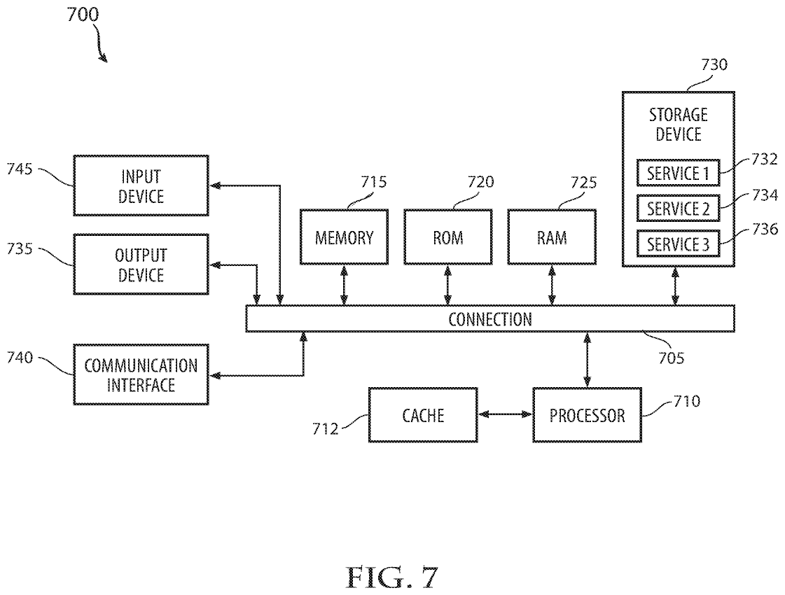

FIG. 7 illustrates example computer devices that can be used in connection with any of the systems disclosed herein. For example, the components of the computer device 700 can be incorporated into aspect of the scale 502 in any respect. In this example, FIG. 7 illustrates a computing system 700 including components in electrical communication with each other using a connection 705, such as a bus. System 700 includes a processing unit (CPU or processor) 710 and a system connection 705 that couples various system components including the system memory 715, such as read only memory (ROM) 720 and random access memory (RAM) 725, to the processor 710. The system 700 can include a cache of high-speed memory connected directly with, in close proximity to, or integrated as part of the processor 710. The system 700 can copy data from the memory 715 and/or the storage device 730 to the cache 712 for quick access by the processor 710. In this way, the cache can provide a performance boost that avoids processor 710 delays while waiting for data. These and other modules can control or be configured to control the processor 710 to perform various actions. Other system memory 715 may be available for use as well. The memory 715 can include multiple different types of memory with different performance characteristics. The processor 710 can include any general purpose processor and a hardware or software service or module, such as service (module) 1 732, service (module) 2 734, and service (module) 3 736 stored in storage device 730, configured to control the processor 710 as well as a special-purpose processor where software instructions are incorporated into the actual processor design. The processor 710 may be a completely self-contained computing system, containing multiple cores or processors, a bus, memory controller, cache, etc. A multi-core processor may be symmetric or asymmetric.

To enable user interaction with the device 700, an input device 745 can represent any number of input mechanisms, such as a microphone for speech, a touch-sensitive screen for gesture or graphical input, keyboard, mouse, motion input, speech and so forth. An output device 735 can also be one or more of a number of output mechanisms known to those of skill in the art. In some instances, multimodal systems can enable a user to provide multiple types of input to communicate with the device 700. The communications interface 740 can generally govern and manage the user input and system output. There is no restriction on operating on any particular hardware arrangement and therefore the basic features here may easily be substituted for improved hardware or firmware arrangements as they are developed.

Storage device 730 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 725, read only memory (ROM) 720, and hybrids thereof.

The storage device 730 can include services or modules 732, 734, 736 for controlling the processor 710. Other hardware or software modules are contemplated. The storage device 730 can be connected to the system connection 705. In one aspect, a hardware module that performs a particular function can include the software component stored in a computer-readable medium in connection with the necessary hardware components, such as the processor 710, connection 705, output device 735, and so forth, to carry out the function.

In some cases, such a computing device or apparatus may include a processor, microprocessor, microcomputer, or other component of a device that is configured to carry out the steps of the methods disclosed above. In some examples, such computing device or apparatus may include one or more antennas for sending and receiving RF signals. In some examples, such computing device or apparatus may include an antenna and a modem for sending, receiving, modulating, and demodulating RF signals, as previously described.

The components of the computing device can be implemented in circuitry. For example, the components can include and/or can be implemented using electronic circuits or other electronic hardware, which can include one or more programmable electronic circuits (e.g., microprocessors, graphics processing units (GPUs), digital signal processors (DSPs), central processing units (CPUs), and/or other suitable electronic circuits), and/or can include and/or be implemented using computer software, firmware, or any combination thereof, to perform the various operations described herein. The computing device may further include a display (as an example of the output device or in addition to the output device), a network interface configured to communicate and/or receive the data, any combination thereof, and/or other component(s). The network interface may be configured to communicate and/or receive Internet Protocol (IP) based data or other type of data.

The methods discussed above can be illustrated as a logical flow diagram, the operations of which represent a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

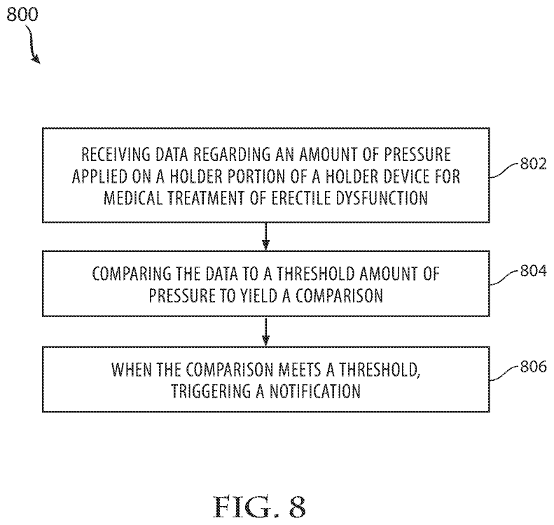

For example, FIG. 8 illustrates a method which can be performed by the scale 502 disclosed above. The method can include receiving data regarding an amount of pressure applied on a holder portion of a holder device for medical treatment of erectile dysfunction (802), comparing the data to a threshold amount of pressure to yield a comparison (804), and when the comparison meets a threshold, triggering a notification (806). Other method elements can include transmitting information from a scale 502 of the holder device 200 to a remote device such that the remote device can take further action, report on the information, receive user input, provide output, aggregate the data, and so forth.

Additionally, the methods disclosed herein may be performed under the control of one or more computer systems configured with executable instructions and may be implemented as code (e.g., executable instructions, one or more computer programs, or one or more applications) executing collectively on one or more processors, by hardware, or combinations thereof. As noted above, the code may be stored on a computer-readable or machine-readable storage medium, for example, in the form of a computer program comprising a plurality of instructions executable by one or more processors. The computer-readable or machine-readable storage medium may be non-transitory.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description above to provide a thorough understanding of the embodiments and examples provided herein. However, it will be understood by one of ordinary skill in the art that the embodiments may be practiced without these specific details. For clarity of explanation, in some instances the present technology may be presented as including individual functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual embodiments may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but can have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

We claim:

1. A device comprising:
a base;
a riser attached to the base at a central portion of the base, the riser comprising a holder support and an open channel through an upper portion of the riser;
a holder having an attachment portion complementary to the holder support in the riser and a holder portion that extends horizontally from a top edge of the attachment portion, wherein the attachment portion comprises a slot and wherein an attachment portion front edge is in a same plane as holder portion front edge;
a first member configured to go through the open channel and the slot; and
a second member attached to the first member and used to lock the holder in a chosen position relative to the riser.

2. The device of claim 1, wherein the base is approximately 15 inches wide, and approximately 4 inches deep.

3. The device of claim 1, wherein the base comprises a cavity that is used to attach the riser to the base via a dowel.

4. The device of claim 1, wherein the riser is approximately 8 inches high.

5. The device of claim 1, wherein the first member and the second member comprise a quick release mechanism.

6. The device of claim 1, wherein the attachment portion of the holder is approximately 5 inches long.

7. The device of claim 1, wherein the holder portion is approximately 7 inches long.

8. The device of claim 1, wherein the holder portion is configured to be approximately 2.2 inches wide and approximately 1 inch tall.

9. The device of claim 8, wherein the holder portion has an interior surface having a first surface at an approximately 90 degree angle to a second surface.

10. The device of claim 1, wherein the holder portion is attached to the attachment portion at a first end, wherein the holder portion comprises a second end having a convex surface of approximately 0.6 inches.

11. The device of claim 1, wherein the device comprises a penile holder device for a medical treatment.

12. The device of claim 1, wherein the riser is cylindrical in shape.

13. The device of claim 1, wherein the device as adjustable to a height of approximately 12.5 inches.

14. The device of claim 1, wherein the holder can adjust vertically relative to the riser or at a tilted angle relative to the riser.

15. The device of claim 1, further comprising:
a scale configured to trigger a notification upon a threshold pressure being pressed down on the holder.

16. The device of claim 15, wherein the scale is configured in the riser.

17. The device of claim 16, wherein the scale is configured in the riser below the holder support.

18. The device of claim 15, further comprising:
a battery that powers the scale; and
a notification component which is triggered upon the threshold pressure being pressed down on the holder.

19. The device of claim 18, wherein the threshold pressure comprises a range of pressures that will trigger the notification component.

20. A device comprising:
a base;
a riser attached to the base at a central portion of the base, the riser comprising a holder support and an open channel through an upper portion of the riser;
a holder having an attachment portion complementary to the holder support in the riser and a holder portion that extends horizontally from a top edge of the attachment portion; an attachment component that adjustably attaches the holder to the riser; and
a scale that triggers a notice upon a threshold pressure being pressed on the holder.

21. The device of claim 20, wherein the scale further comprises a battery and an electronic notification component, wherein the notice comprises one of an audio notice, a visual notice or a tactile notice.

22. The device of claim 20, wherein the scale further comprises a controller that is configured to adjust the threshold pressure on an individual basis.

23. A method comprising:
receiving data regarding an amount of pressure applied on a holder portion of a holder device for medical treatment of erectile dysfunction;
comparing the data to a threshold amount of pressure to yield a comparison; and
when the comparison meets a threshold, triggering a notification.
